(19) United States
(12) Patent Application Publication
(10) Pub. No.: US 2009/0048557 A1
(43) Pub. Date: Feb. 19, 2009

(54) DEVICE AND METHODS COMBINING VIBRATING MICRO-PROTRUSIONS WITH PHOTOTHERAPY

(76) Inventors: Yehushua Yeshurun, Haifa (IL); Yotam Levin, Nes Ziona (IL); Yotam Almagor, Jerusalem (IL); Yoel Sefi, Merom Hagalil (IL); Lior Wolloch, Kiryat Motzkin (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/296,129

(22) PCT Filed: Apr. 19, 2007
(86) PCT No.: PCT/IL07/00492

§ 371 Date: Oct. 6, 2008

Related U.S. Application Data
(60) Provisional application No. 60/807,072, filed on Jul. 12, 2006.
(60) Provisional application No. 60/745,191, filed on Apr. 20, 2006.

(51) Int. Cl. A61B 17/00; A61N 5/06; A61M 35/00

(52) U.S. Cl. 604/20; 606/131; 607/88

(57) ABSTRACT

Method and device for therapeutic or aesthetic treatment of the skin performs a combination of dermabrasion to mechanically modify at least an outer layer of the skin in a first region and phototherapy. The device preferably employs a skin interface element with projections protruding from a substrate which is moved in a vibratory motion by a vibration generating mechanism. This is combined with an illumination system deployed to direct a therapeutically relevant dosage of light towards a surface of the skin against which the skin interface unit is in contact.

### FIELD AND BACKGROUND OF THE INVENTION

The present invention relates to skin treatment and, in particular, it concerns devices and methods combining vibrating micro-protrusions with phototherapy.

Conventional micro-dermabrasion abrades the skin with a high-pressure flow of crystals. Micro-dermabrasion was developed in Italy in 1985; its use was widespread in European countries prior to its introduction and popularity in the United States. This technology offers the advantages of low risk and rapid recovery compared with more traditional resurfacing modalities such as laser and can be effective in the appropriate patient population. Abrading the superficial layers of the skin induces cellular proliferation and production of various elements of the extracellular matrix, resulting in an improved aesthetic appearance of the skin. Providing this process is contained to the epidermis and shallow dermis, scarring is unlikely. Additional modalities for dermal abrasion include traditional dermabrasion, chemical peeling, laser resurfacing. All of these resurfacing procedures exert their effects through different degrees of epidermal and dermal ablation. The results and the indications for each modality depend on the depth of ablation. These modalities are invariably painful and provide significant local side effects such as erythema, edema, itching, burning sensation, sun sensitivity and the like.

Photomodulation refers to the use of low energy, narrow-band light with or without a specific pulse (on/off) sequence. Photomodulation has been shown to regulate cell activity. This process is relatively new and differs from conventional methods in that it is non-thermal. Light emitting diode (LED) light sources are frequently used for photomodulation, which has been shown to improve photoaging skin changes, facial texture, fine lines, background erythema, pigmentation and wound healing. Photomodulation is thought to be produced, among others, by the activation (and increased energy levels) of light-sensitive organelles (sensors) present in skin cells and by the generation of ROS (Reactive Oxygen Species). The effects of photomodulation can be further enhanced by introducing substances (e.g., creams) that become active when exposed to light into the treated area (photodynamic therapy). In certain specific indications such as acne, phototherapy can be performed by projecting blue light, which is phototoxic to the acne bacteria.

Other modes of phototherapy for treatment of the skin employ higher energy radiation, usually in the infrared part of the spectrum, for delivering heat to selected layers of the skin. Common examples include the use of CO₂ lasers (wavelength about 10,000 nm) or Er:YAG lasers (wavelength about 3,000 nm) for peeling or skin resurfacing in the epidermis or a portion thereof, and the use of Nd:YAG lasers (wavelength of about 1,000-1,500 nm) which is less absorbed by superficial layers of the skin and penetrates deeper, performing applications such as tattoo removal, hair removal, acne treatment etc.

In any and all of the aforementioned modes of phototherapy where the target layer of tissue is not the most superficial layer of the skin, energy losses occur to varying degrees through absorption, scattering or reflection of radiation by the higher layers, thereby reducing the efficacy of the process.

There is therefore a need for a method and device which would employ a combination of dermabrasion and phototherapy to advantage, optionally with delivery of additional therapeutic substances. It would also be advantageous to improve the efficacy of a phototherapy treatment by employing dermabrasion to reduce the barrier effect of at least one outer layer of the skin to penetration of the corresponding radiation.

### SUMMARY OF THE INVENTION

The present invention is a method and device for performing a combination of dermabrasion and phototherapy.

According to the teachings of the present invention there is provided, a method for treatment of the skin comprising implementing a combined treatment including substantially contemporaneous or sequential steps of: (a) performing dermabrasion to mechanically modify at least an outer layer of the skin in a first region; and (b) delivering to the first region a therapeutically relevant dosage of light so as to perform phototherapy.

According to a further feature of the present invention, the dermabrasion is performed prior to or during the delivering in such a manner as to reduce obstruction of the light passing through the outer layer of the skin.

According to a further feature of the present invention, the dermabrasion is micro-dermabrasion.

According to a further feature of the present invention, the dermabrasion is performed by bringing a plurality of micro-protrusions into contact with the skin and generating vibratory motion of the micro-protrusions.

According to a further feature of the present invention, the micro-protrusions are provided with an antibacterial coating.

According to a further feature of the present invention, at least part of a support structure supporting the micro-protrusions is formed from a material substantially transparent to the light, and wherein at least some of the light is delivered via the substantially transparent material.

According to a further feature of the present invention, during the dermabrasion, a therapeutic substance is delivered to the first region of skin.

According to a further feature of the present invention, the therapeutic substance is an agent for enhancing action of the phototherapy.

There is also provided according to the teachings of the present invention, a device for treatment of the skin comprising: (a) a skin interface element including a substrate provided with a plurality of protrusions; (b) a vibration generating mechanism mechanically linked to the skin interface element so as to generate vibratory motion of the skin interface element; and (c) an illumination system deployed to direct a therapeutically relevant dosage of light towards a surface of the skin against which the skin interface unit is in contact.

According to a further feature of the present invention, there is also provided a housing and a support structure supporting the skin interface element relative to the housing, wherein at least part of at least one of the substrate and the support structure is formed from a material substantially transparent to the light, the illumination system being deployed to direct at least some of the light via the substantially transparent material.

According to a further feature of the present invention, the protrusions project to a height above the substrate of no greater than 200 microns, and preferably between about 20 microns and about 100 microns.

According to a further feature of the present invention, the protrusions are arranged in a two-dimensional array.

According to a further feature of the present invention, the protrusions have a shape selected from the group comprising: pyramids, cones and rods.

According to a further feature of the present invention, the protrusions are integrally formed with the substrate.

According to a further feature of the present invention, the protrusions and the substrate are formed from a single crystal of material.

According to a further feature of the present invention, the protrusions and the substrate are formed from a unitary block of material processed primarily by wet etching techniques.

According to a further feature of the present invention, the protrusions are formed from a material selected from the group consisting of: silicon, a polymer, a metal, a metal alloy, and a ceramic material.

According to a further feature of the present invention, the protrusions are provided with an antibacterial coating.

According to a further feature of the present invention, the vibration generating mechanism includes a motor configured for rotating an eccentric weight about an axis.

According to a further feature of the present invention, the vibration generating mechanism is configured to generate vibratory motion corresponding to an orbital motion in a plane of the substrate.

According to a further feature of the present invention, the vibration generating mechanism is configured to generate vibratory motion having a non-zero component perpendicular to a plane of the substrate.

According to a further feature of the present invention, the vibration generating mechanism is configured to generate vibratory motion having a frequency in the range between 50 Hz and 200 Hz.

According to a further feature of the present invention, the vibration generating mechanism is configured to generate vibratory motion having a frequency in the range of 140 Hz±25 Hz.

According to a further feature of the present invention, there is also provided a pressure-limiting switch arrangement associated with the skin interface element and responsive to contact pressure of the skin interface element above a given limit to interrupt operation of the vibration generating mechanism.

According to a further feature of the present invention, there is also provided a housing, wherein the skin interface element is resiliently mounted relative to the housing, and wherein the vibration generating mechanism is mechanically linked to the skin interface element so as to generate vibratory motion of the skin interface element relative to the housing.

According to a further feature of the present invention, there is also provided a housing mechanically supporting the skin interface element, the vibration generating mechanism, the illumination system and at least one electric battery, wherein the vibration generating mechanism and the illumination system are powered exclusively by the at least one electric battery.

There is also provided according to the teachings of the present invention, a device for treatment of the skin comprising: (a) a skin interface element including a substrate provided with a plurality of protrusions; and (b) an antibacterial coating applied at least to surfaces of the protrusions.

According to a further feature of the present invention, the antibacterial coating is applied to a surface of the substrate.

According to a further feature of the present invention, the plurality of projections are implemented as a plurality of hollow microneedles.

According to a further feature of the present invention, the antibacterial coating includes titanium dioxide.

According to a further feature of the present invention, the antibacterial coating includes metal ions of at least one metal selected from the group consisting of: silver, zinc, cobalt, aluminum, mercury and copper.

According to a further feature of the present invention, the antibacterial coating includes benzalkonium chloride.

### BRIEF DESCRIPTION OF THE DRAWINGS

The invention is herein described, by way of example only, with reference to the accompanying drawings, wherein:

FIG. 1 is a schematic representation of a device for combined dermabrasion and phototherapy of the skin, constructed and operative according to the teachings of the present invention;

FIG. 2 is a schematic representation of a variant of the device of FIG. 1 illustrating an additional pressure-limiting switch arrangement;

FIG. 3 is an isometric view of a first implementation of a skin interface element from the device of FIG. 1;

FIG. 3A is an enlarged view of a single micro-protrusion from the skin interface element of FIG. 3;

FIG. 4 is an isometric view of a second implementation of a skin interface element from the device of FIG. 1;

FIG. 4A is an enlarged view of a single micro-protrusion from the skin interface element of FIG. 4;

FIG. 5 is an isometric view of an implementation of the device of FIG. 1;

FIG. 6 is an enlarged view of a part of FIG. 5 showing a skin interface element;

FIG. 7 is an enlarged view of a small region of FIG. 6 showing the structure of the protrusions; and

FIG. 8 is a partially cut-away isometric view of a part of the device of FIG. 5.

### DESCRIPTION OF THE PREFERRED EMBODIMENTS

The present invention is a method and device combining dermabrasion with phototherapy.

The principles and operation of methods and devices according to the present invention may be better understood with reference to the drawings and the accompanying description.

Before turning to specific examples of preferred devices according to the present invention, it should be appreciated that the invention is applicable generally to any and all combined techniques in which dermabrasion and phototherapy are performed substantially contemporaneously or sequentially in such a manner that they provide additive or synergistic therapeutic or aesthetic results.

Thus, in general terms, the present invention provides a method for treatment of the skin implementing a combined treatment including substantially contemporaneous or sequential steps of: (a) performing dermabrasion to mechanically modify at least an outer layer of the skin in a first region; and (b) delivering to the first region a therapeutically relevant dosage of light so as to perform phototherapy. According to one particularly significant subset of applications, the dermabrasion is performed prior to or during the delivering of light in such a manner as to reduce obstruction of the light passing through the outer layer of the skin, thereby improving efficacy of the phototherapy. Additionally, or alternatively, a therapeutic substance is delivered to the first region of skin during the dermabrasion. The therapeutic substance may be a medication for performing a complementary or independent therapeutic function, and according to one particularly preferred example, may be an agent for enhancing action of the phototherapy or whose action is actuated or enhanced by the light administered during the phototherapy.

According to most preferred implementations, the dermabrasion, which is preferably implemented as micro-dermabrasion, is performed by bringing a plurality of micro-protrusions into contact with the skin and generating vibratory motion of the micro-protrusions. The use of microprotrusions has also been found to give rise to localized heating of the skin near the microprotrusions' tips. This heating is also believed to contribute to tissue regeneration, providing a further synergistic effect. Various features of preferred devices for performing both dermabrasion and phototherapy will be described in detail below.

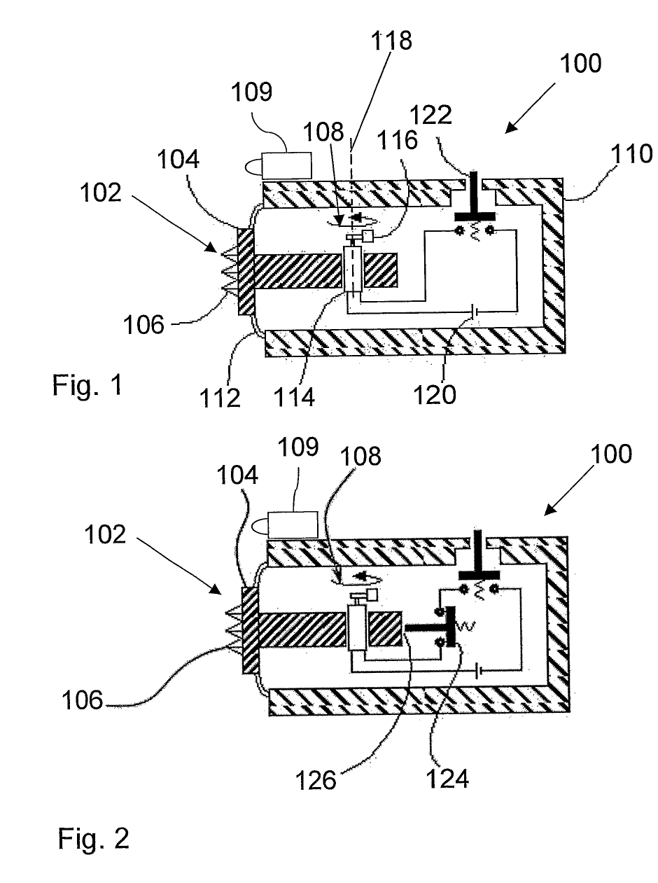

Referring now to the device of the present invention, FIG. 1 illustrates schematically a device, generally designated 100, constructed and operative according to the teachings of the present invention, for therapeutic treatment of the skin. Generally speaking, device 100 includes a skin interface element 102 having a substrate 104 from which project a plurality of protrusions 106. A vibration generating mechanism 108 is mechanically linked to skin interface element 102 so as to generate vibratory motion of skin interface element 102. An illumination system 109 is deployed to direct a therapeutically relevant dosage of light towards a surface of the skin against which the skin interface unit is in contact.

The combination of the vibratory motion together with the protrusions 106 achieves superficial abrasion of the outer surface of the skin, typically only at the level of the stratum corneum (SC) or, in relevant specific cases, the epidermis, and is thus effective for a wide range of cosmetic and medical applications as are known for micro-dermabrasion or dermabrasion. This dermabrasion in combination with phototherapy performed by illumination system 109 provides an additive and/or synergistic therapeutic or aesthetic effect, as will be described. These and other advantages of the present invention will become clearer from the following description.

Before addressing the features of the present invention in more detail, it will be helpful to define certain terminology as used herein in the description and claims. Firstly, reference is made to “vibration” and “vibratory motion”. These terms are used herein in the description and claims to refer to any repetitive oscillatory motion about a mean position in one or more dimension. These vibrations may be linear (i.e., one dimensional) or orbital (i.e., circular or elliptical), or may have a more complex form such as results from, for example, differing vibration frequencies in two perpendicular directions. The vibratory motion is preferably translational rather than rotating. In other words, the motion of all parts of skin interface element 102 is preferably roughly the same so that the entire element vibrates to-and-fro, or orbits, without overall rotation of skin interface element 102.

Typically, the vibrations are actually oscillating forces applied to the skin interface element 102 and the amplitude of the vibrations varies, depending upon the damping effect of engagement with the skin.

The term “superficial” is used herein in the description and claims to refer to abrasion of the skin which does not extend to a depth of more than 200 microns. For cosmetic applications, the superficial abrasion is preferably kept to a depth less than 100 microns, thereby avoiding fully breaching the upper barrier layers of the skin (stratum corneum and upper epidermal layers), so as to minimize pain, damage to the viable dermis, and other adverse effects. For certain particularly preferred applications, an abrasion depth of roughly 10-20 microns may be preferred in order to breach the stratum corneum (SC) alone without damaging living cells. For this purpose, penetration into the layers of the skin is preferably limited to less than 100 microns, and most preferably less than about 70 microns. The actual height of the protrusions 106 above the surface of substrate 104 may be somewhat larger than the desired penetration depth, since the entire height does not typically penetrate. Preferred heights for protrusions 106 are thus typically in the range of about 20 microns to about 100 microns, and most preferably 60 microns±20 microns. For medical applications, on the other hand, penetration depths in excess of 100 microns are typically indicated. In this case, protrusions of height in the range of 100 microns up to 200 microns are typically used, although taller protrusions up to about 500 microns could also be useful in certain applications.

The term “dermabrasion” is used herein generically to refer to any and all techniques in which skin is mechanically abraded, independent of depth. The term thus defined includes the special case of “micro-dermabrasion” in which the abrasion is performed to a depth of no more than 200 microns.

The term “protrusions” is used to refer to any repetitive structure of projecting features which project from the surface of substrate 104. The protrusions may be any shape, pointed or blunt-ended, rounded in cross-section or with lateral cutting edges, hollow or solid. Non limiting examples of particularly preferred forms of protrusion include: symmetrical or asymmetric pyramids of polygonal base, pointed or truncated cones, and cylindrical or polygonal rods. The protrusions of the present invention are referred to interchangeably as “micro-protrusions” in view of their preferred ranges of dimensions under 500 microns as described above.

The term “phototherapy” is used herein as a generic term to refer to any and all processes in which visible or invisible (IR or UV) light is used for therapeutic or aesthetic purposes on the skin or any other surface of a human or animal body. Phototherapy thus defined encompasses a wide range of different light-based techniques including, but not limited to, photomodulation, photodynamic therapy (PDT) and photoablation.

The phrase “therapeutically relevant” is used herein in the description and claims referring to application of light to denote a process which, under the conditions employed, produces or causes a change in the tissue to which it is applied. The change may be conditional upon the presence of additional substances (e.g., a photo-sensitizer) and may not be readily or immediately discernable. The phrase “therapeutically relevant” is used to distinguish the present invention from illumination arrangements which are designed and employed only for improving visibility or as part of an imaging system.

Finally with regard to terminology, to specify the temporal relation between the dermabrasion and phototherapy of the present invention, these processes are described as being performed “substantially contemporaneously” or “sequentially”. The phrase “substantially contemporaneously” is used herein to refer to processes being performed with some degree of temporal overlap. This includes possibilities of both processes being performed simultaneously, of one being performed during a time slot within the duration of the other, and of a later process starting during the duration of the former process and continuing after the former process has stopped. The term “sequentially” is used herein to refer to two processes which are performed without temporal overlap, but within a sufficiently short time period of each other that the effect of the earlier process is still present in order for the later process to be additive or synergistic in its effects.

Turning now to the features of the present invention in more detail, protrusions 106 are preferably arranged in a two-dimensional array, and typically in a rectangular array, i.e., with protrusions spaces along two perpendicular directions. The area covered by the array is not necessarily, or even typically, rectangular, and roughly round or octagonal areas may have advantages in terms of symmetry of coverage and accessibility to hard-to-reach regions of the skin.

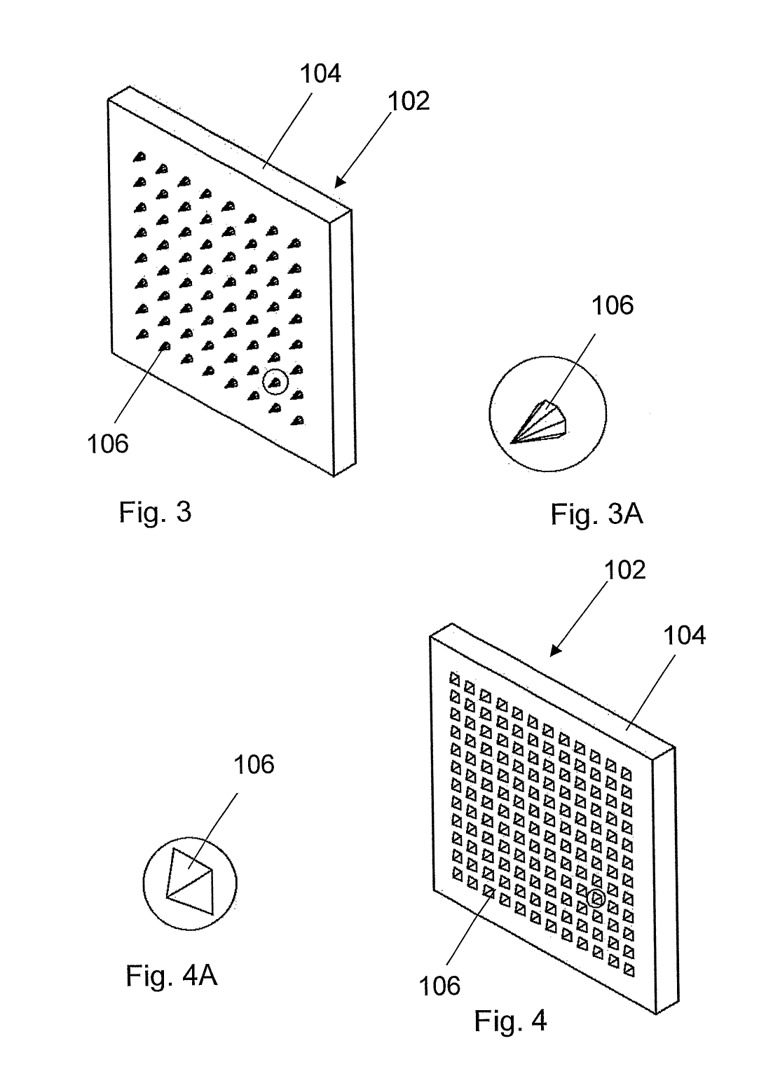

Two non-limiting examples of arrangements of protrusions are illustrated in FIGS. 3 and 4. Most preferably, dimensions of the two-dimensional array are at least 8×8, and more preferably at least 10×10, corresponding to a total of at least 100 protrusions. Typically, several hundred protrusions are provided on an area of less than one square centimeter. In the examples illustrated here, FIG. 3 (enlarged in FIG. 3A) shows octagonal pyramid protrusions, while FIG. 4 (enlarged in FIG. 4A) shows square pyramidal protrusions. In either case, the protrusions may optionally be modified by truncation to form a stronger but less sharp form.

Protrusions 106 may be produced using a wide range of different technologies from a wide variety of different materials. For example, MEMS technology (using wet or dry etching or a combination of the two) may be employed to process a unitary block of silicon (single crystal) or other etchable material to produce the protrusions-plus-substrate structure. Suitable MEMS techniques for forming a wide variety of conical, pyramidal and cylindrical protrusions projecting from a substrate are well known in the art, for example, in the context of microneedle technology. Most preferably, low cost MEMS techniques based primarily on wet etching techniques are used.

Other technologies suitable for forming the skin interface element include injection or micro-injection molding, hot embossing and machining techniques which be used to produce the skin interface element from various polymers or other moldable materials. According to a further option, foils (such as steel, titanium, or other metals or metal alloys) may be processed by cutting (wire cutting, laser cutting, punching or other cutting processes), with or without post cutting processing, to form protrusions 106. Ceramics may also be used. In most preferred implementations, protrusions 106 are integrally formed with substrate 104.

Referring again to FIG. 1, skin interface element 102 is preferably supported relative to a housing 110 via a resilient support 112 which allows vibratory motion of skin interface element 102 without excessive damping from the mass of housing 110 and the user's hand holding the device. The isolation of most of the vibrational energy from the main body of housing 110 also serves to improve user comfort and renders the device more energy efficient. In the case of FIG. 1, resilient support 112 is shown as a flexible membrane which performs an additional function of sealing between skin interface element 102 and housing 110 to prevent ingress of dirt and other foreign matter. Resilient support 112 may be formed of any suitable resilient material, including but not limited to, natural or artificial rubber or silicone.

Generation of vibration can be achieved using any of a wide range of mechanisms. By way of one preferred but non-limiting example, vibration generating mechanism 108 as illustrated here includes an electric motor 114 driving an eccentric weight 116 about an axis 118. The motor is driven by a power supply 120, typically implemented as one or more battery mounted within housing 110, and is controlled by on/off switch 122. Vibration generating mechanism 108 can thus be implemented cheaply using compact off-the-shelf components such as those employed for vibrating notification in cellular telephones. Alternatively the vibration generating mechanism can be implemented as a piezoelectric crystal or a solenoid.

Although on/off switch 122 is illustrated here as a simple on/off push-button switch, it should be noted that alternative electrical switch arrangements and/or electronic control circuitry may be used to advantage to provide various modes of control over the device. By way of non-limiting examples, modes of actuation may include one or more of the following:
Bistable on/off switch (slider or push-button) manually actuated by the user to switch on and manually actuated by the user to switch off,
Hold-on switch requiring continuous pressure from the user to maintain operation of the device and switching off when released. This switch system provides the highest safety and energy saving.
Push button switch with timer in which the user activates the device and the operation stops automatically after a pre-set time. This mode of operation enables a controlled treatment durations and reduces risks of over usage (irritation). The end of operation may be indicated by a buzzer or the like. Optionally, the timer may be controllable to operate for different periods, suitable for different modes of treatment (for example, for different skin sites).
Optionally, operation may be made conditional on a predefined minimum and/or maximum contact pressure between the device and the skin. An example of a mechanism for cutting out operation in the case of excess contact pressure with the skin is described below with reference to FIG. 2.
In each of the above cases, an indicator light may be provided to indicate when the device is operating. Optionally, the same indicator light may be used to indicate low battery, for example, through flashing.

The deployment of the vibration generating mechanism and its attachment to the other parts of the device are chosen relative to the micro-protrusions in order to provide a desired form of vibrational motion relative to the skin surface (e.g., orbital motion on the skin, motion perpendicular to the skin, a back and fro motion on the skin, or any combination of these motions). Thus, for example, in the case illustrated here, axis 118 is substantially parallel to the surface of substrate 104, resulting in vibratory motion having a first component parallel to the skin surface and a second (non-zero) component perpendicular to the skin surface. Alternatively, axis 118 may be deployed perpendicular to the surface of substrate 104, resulting in a rotating force vector in a plane of the substrate and a corresponding orbital motion of skin interface element 102. A preferred non-limiting range of frequencies for the vibration generating mechanism is between 50 Hz and 200 Hz, and most preferably, in the range of 140 Hz±25 Hz.

The application times for the dermabrasion treatment are preferably less than 1 minute, and most preferably in the range of 10-35 seconds. The following experimental data resulted from tests performed to evaluate risks of irritation through the operation of the dermabrasion aspect of the present invention, and show the action of the device to be non-irritating as follows.

Experimental Procedure: The device was used to abrade small areas of skin, app. 1 square cm in size on a daily basis for a period of 26 days. 3 sites were chosen on each arm, in the volar aspect, and one site on each temple. Every site was abraded daily for a predetermined duration as follows:

On each hand, one site was abraded for 5 seconds, one for 10 seconds and one for 30 seconds. On the right temple, the site was abraded for 5 seconds and on the left temple for 10 seconds.

The contact force applied was approximately 3N. The vibrating motion was mostly radial, i.e., generally parallel to the skin plane. The application included circular motion as well.

Local irritation was assessed daily before abrasion using the Draize score (Draize J H. “Dermal and eye toxicity tests” Principles and procedures for evaluating the toxicity of household substances. Washington, D.C.: National Academy of Sciences, 1997:31-2), as detailed below.

Results are described in the following table:

| Day | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
|---|---|---|---|---|---|---|---|---|---|---|---|---|---|---|---|---|---|---|---|---|---|---|---|---|---|---|
| RA5 | 0 | 0 | 0 | 0 | 0 | 0 | 0 | 0 | 0 | 0 | 0 | X | 0 | 0 | 0 | 0 | 0 | 0 | 0 | 0 | 0 | 0 | X | 0 | 0 | 0 |
| RA10 | 0 | 0 | 0 | 0 | 0 | 0 | 0 | 1 | 1 | 0 | 0 | X | 0 | 0 | 0 | 0 | 0 | 0 | 0 | 0 | 0 | 0 | X | 0 | 0 | 0 |
| RA30 | 0 | 0 | 0 | 0 | 0 | 0 | 0 | 1 | 1 | 0 | 0 | X | 0 | 0 | 0 | 1 | 1 | 1 | 0 | 0 | 0 | 0 | X | 0 | 0 | 0 |
| RT5 | 0 | 0 | 0 | 0 | 0 | 0 | 0 | 0 | 0 | 0 | 0 | X | 0 | 0 | 0 | 0 | 0 | 0 | 0 | 0 | 0 | 0 | X | 0 | 0 | 0 |
| LA5 | 0 | 0 | 0 | 0 | 0 | 0 | 0 | 0 | 0 | 0 | 0 | X | 0 | 0 | 0 | 0 | 0 | 0 | 0 | 0 | 0 | 0 | X | 0 | 0 | 0 |
| LA10 | 0 | 0 | 0 | 0 | 0 | 0 | 0 | 0 | 0 | 0 | 0 | X | 0 | 0 | 0 | 0 | 0 | 0 | 0 | 0 | 0 | 0 | X | 0 | 0 | 0 |
| LA30 | 0 | 0 | 0 | 0 | 0 | 0 | 0 | 1 | 0 | 0 | 0 | X | 0 | 0 | 0 | 1 | 1 | 1 | 1 | 0 | 0 | 0 | X | 0 | 1 | 1 |
| LT10 | 0 | 0 | 0 | 0 | 0 | 0 | 0 | 1 | 0 | 0 | 0 | X | 0 | 0 | 0 | 1 | 1 | 1 | 0 | 0 | 0 | 0 | X | 0 | 1 | 1 |

Index: RA5—Right Arm 5 seconds/day
RA10—Right Arm 10 seconds/day
RA30—Right Arm 30 seconds/day
RT5—Right Temple 5 seconds/day
LA5—Left Arm 5 seconds/day
LA10—Left Arm 10 seconds/day
LA30—Left Arm 30 seconds/day
LT10—Left Temple 10 seconds/day
Numbers in Bold indicate the presence of mild local desquamation (which is not assessed in the Draize score). X marks a day on which the treatment was missed.

The Draize score is defined as follows:

| | | | | Hemorrhage/ |
|---|---|---|---|---|
| Score | Erythema | Edema | Pruritus | Petechiae |
| 0 | No erythema | No edema | No pruritus | None |
| 1 | Very slight erythema | Very slight edema | Occasional | Isolated, up to 5 |
| | (barely perceptible) | (barely | pruritus | petechiae |
| | | perceptible) | | |
| 2 | Well-defined | Slight edema | Constant | Isolated but >5 |
| | erythema | (edges of area | pruritus | petechiae |
| | | well defined by | | |
| | | slight raising) | | |
| 3 | Moderate to severe | Moderate edema | NA | Many, with some |
| | erythema | (raised >1 mm) | | coalescence |
| 4 | Severe erythema (beet | Severe edema | NA | Numerous petechiae, |
| | redness) to slight | (raised >1 mm, | | with or without |
| | eschar formation | extending beyond | | pinprick spots of |
| | (injuries in depth) | area of exposure) | | blood on surface |
| 5 | NA | NA | NA | Frank bleeding |

NA indicates “not applicable”.

Illumination system 109 may be any illumination system generating light of wavelength(s) and intensity suitable for implementing the desired type of phototherapy. By way of non-limiting example, the device of the present invention is illustrated herein in the context of a hand-held battery-powered device suitable for performing various types of photomodulation. In this case, one or more light emitting diode (LED) of suitable wavelength is preferably used. Other types of phototherapy, including high intensity light application, may also be implemented according to the teachings of the present invention, typically using a device directly connected to an external electrical power source of suitable rating. In each case, illumination system 109 is preferably implemented with a built-in electronic control unit for controlling current supply, power, and the operational duty cycle to implement the desired phototherapy.

As mentioned, the type of light source and operational parameters are chosen according to the specific field of implementation. For example, current scientific literature suggests that light in the blue spectrum (˜410 nm) is specifically effective for treating acne (with or without the combination of photodynamic therapy), while the yellow spectrum (590 nm) is specifically good for skin rejuvenation. Light in the red and near infrared (NIR) spectrum (670-880 nm) is particularly useful for inducing wound and tissue repair.

The mechanical abrasion caused by the vibration of the mechanical skin interface (microprojections) is effective in certain cases to enhance or augment the delivery of light energy into the skin, either by allowing it to be delivered more deeply into the tissue, or provide higher energy levels to the same tissue depth (or a combination of the two effects).

Various applications of the invention combine the device with a specific active composition (cream, gel, solution or the like). For example, the device may be used as a cosmetic or dermatologic pre-treatment, prior to application of an active composition, as a cosmetic or dermatologic post treatment after the cream, paste or solution were applied on treated site, or concurrently during application of a cosmetic or dermatologic treatment.

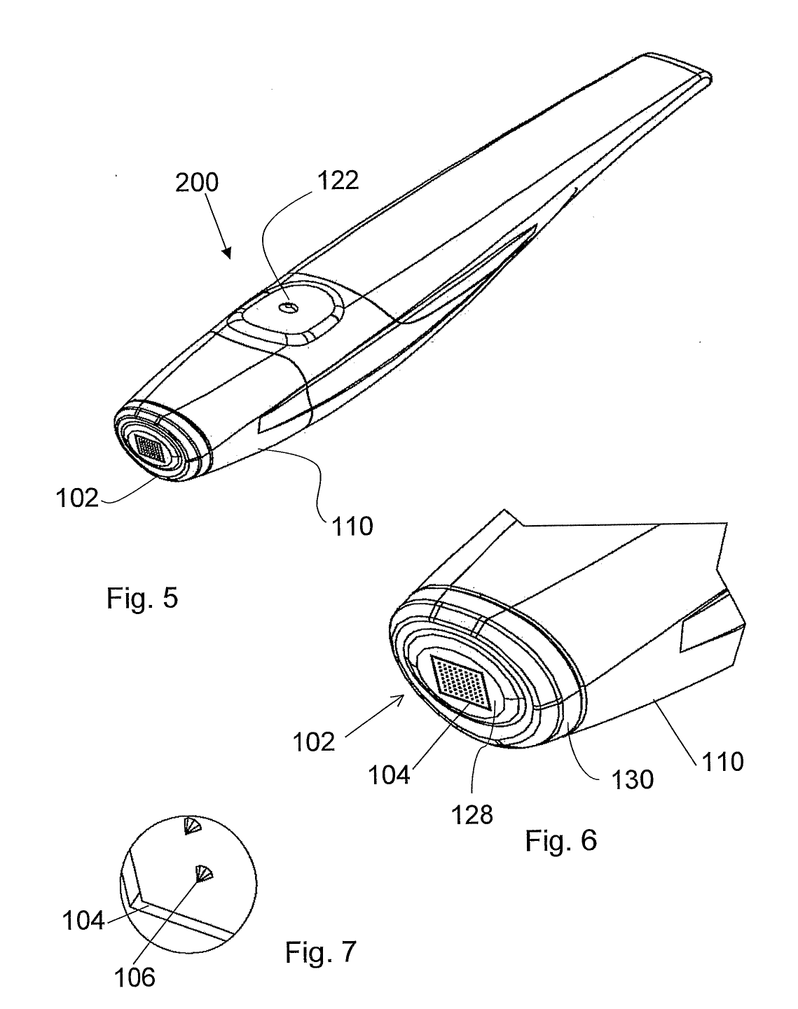

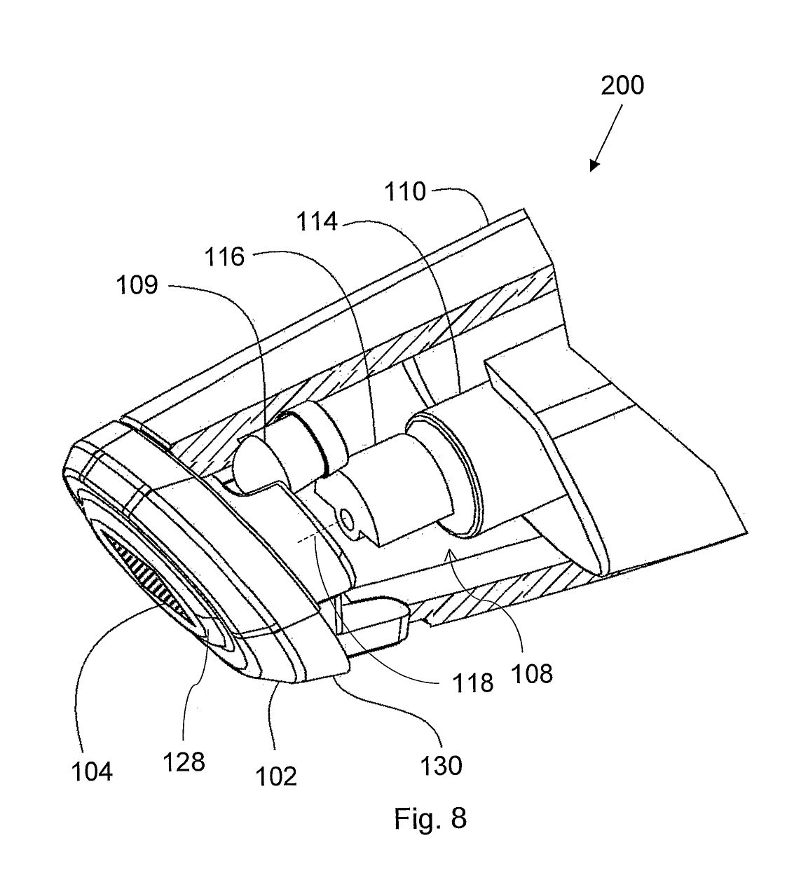

In the schematic illustration of FIG. 1, illumination system 109 is shown supported on an external surface of housing 110. Such a location is possible in a practical implementation, either illuminating the region of skin subject to dermabrasion from the side or alternatively illuminating a region adjacent to the region currently undergoing dermabrasion so that the phototherapy and dermabrasion occur sequentially as the device is moved across the skin. An alternative implementation for simultaneous administration of the two processes will be described below with reference to FIGS. 5-8.

A further feature of certain preferred embodiments of the present invention is illustrated schematically in FIG. 2. FIG. 2 shows a device, generally designated 100′, which is equivalent to device 100 of FIG. 1 except that it features an additional pressure-limiting switch arrangement 124. Pressure-limiting switch arrangement 124 is responsive to contact pressure of skin interface element 102 above a given limit to interrupt operation of the vibration generating mechanism 108. This ensures that contact pressure exerted by the hand of the user does not reach sufficient levels to cause excessive penetration depth, or to lodge protrusions 106 firmly into the tissue, an effect which might lead to excessive damping of vibrations and consequent disruption to the efficacy of the abrasion treatment. This feature is particularly important for medical application (e.g., treatment to increase porosity of the skin to enhance absorption of medication) where relatively longer protrusions may be used and regulation of penetration depth therefore becomes more important.

Structurally, pressure-limiting switch arrangement 124 is shown here implemented as a circuit breaker included in the power supply circuit for vibration generating mechanism 108. The resilient mounting of skin interface element 102 allows for retraction of the skin interface element as a function of contact pressure. By leaving an appropriately chosen gap between the rear end of a shaft 126 of skin interface element 102 and the circuit breaker, a desired threshold of contact pressure can be defined for the cut-out function. Optionally, pressure-limiting switch arrangement 124 may be configured to operate an alarm or buzzer (not shown) if the contact pressure exceeds the defined limit. For most applications, preferred contact force is in the range of 1-5 N, and most preferably around 3 N. The cut-out function can thus advantageously be configured to cut out operation of the device when a threshold chosen in the range of about 3-6 N is exceeded.

Turning now to FIGS. 5-8, these illustrate one non-limiting practical implementation of a device 200 constructed and operative according to the teachings of the present invention. Device 200 is essentially similar to device 100 illustrated schematically in FIG. 1, with equivalent elements labeled similarly. However, the implementation shown here illustrates a number of additional preferred features which were either omitted or simplified for clarity in the schematic representation of FIG. 1. These features will now be addressed.

Most notably, illumination system 109 is here implemented as a hidden illumination system located within housing 110, as best seen in FIG. 8. In order to enable delivery of the light generated by illumination system 109 to the skin at or near skin interface element 102, at least part of substrate 104 and/or of a support structure 128 around the substrate is formed from a material substantially transparent to light of the wavelengths generated. In the preferred example illustrated here, a major part and typically the entirety of support structure 128 is formed from medical/optical grade transparent polymer by common technologies such as injection molding. An example of a polymer material with suitable properties is polycarbonate which provides an excellent combination of biocompatibility and optical performance. According to a particularly preferred optional feature of the present invention, for cases in which substrate 104 is opaque to the wavelengths of illumination used, the transparent material is formed as a lens configured to guide the light around the substrate and towards part of the surface of the skin which would otherwise be obscured by the substrate.

According to an additional, or alternative, approach, transmission of light may be effected at least in part via openings formed through the substrate. In some cases, an opening may be associated with each projection, such as in the case of microprojections which are hollow or otherwise formed with a through-channel.

In certain preferred embodiments such as illustrated here, skin interface element 102 is implemented as part of a replaceable, disposable sub-unit 130. This facilitates proper hygiene, enabling all parts of the device coming in contact with the treated area of skin to be new and clean for each use while avoiding unnecessary costs of replacing other parts of the device.

Housing 110 and other parts of the devices of the present invention which do not need to be transparent are preferably formed from common thermoplastic polymers suitable for injection molding, such as for example ABS (Acrylonitrile Butadiene Styrene).

Device 200 is also distinguished from device 100 described above in that motor 114 is here deployed to rotate eccentric weight 116 about an axis 118 substantially perpendicular to the plane of substrate 104, as best seen in FIG. 8. As a result, the vibratory motion generated by the device is primarily orbital motion in the plane of skin contact.

In all other respects, the structure and operation of device 100 will be fully understood by analogy to the structure and operation of device 100 as described above.

Finally, it should be noted that the present invention may be used to advantage in a wide range of cosmetic and medical application. By way of non-limiting examples, various application procedures could be employed in combining the device with a specific active (cream, gel, solution or the like). For example: cosmetic or dermatologic pre-treatment (skin treatment prior to applying the active composition), cosmetic or dermatologic post treatment (using the device after the cream, paste or solution were applied on treated site), and cosmetic or dermatologic treatment: cream and projections applied concurrently. It should be noted that particularly preferred implementations of the present invention relate to general purpose devices which may be used with various different treatment compositions, and wherein the device itself typically does not store or apply the composition.

Any cosmetic and pharmaceutical agents may be incorporated or delivered with the abovementioned systems to enhance the therapeutic effects of those cosmetic and pharmaceutical agents to improve cosmetic conditions or to alleviate the symptoms of dermatologic disorder. Cosmetic and pharmaceutical agents include those that improve or eradicate age spots, keratoses and wrinkles; analgesics; anesthetics; antiacne agents; antibacterials; antiyeast agents; antifungal agents; antiviral agents; antidandruff agents; antidermatitis agents; antipruritic agents; antiemetics; antimotion sickness agents; antiinflammatory agents; antihyperkeratolytic agents; antidryskin agents; antiperspirants; antipsoriatic agents; antiseborrheic agents; hair conditioners and hair treatment agents; antiaging and antiwrinkle agents; antiasthmatic agents and bronchodilators; sunscreen agents; antihistamine agents; skin lightening agents; depigmenting agents; vitamins; corticosteroids; tanning agents; hormones; retinoids; topical cardiovascular agents and other dermatologicals.

Some examples of cosmetic and pharmaceutical agents are clotrimazole, ketoconazole, miconazole, griseofulvin, hydroxyzine, diphenhydramine, pramoxine, lidocaine, procaine, mepivacaine, monobenzone, erythromycin, tetracycline, clindamycin, meclocycline, hydroquinone, minocycline, naproxen, ibuprofen, theophylline, cromolyn, albuterol, retinoic acid, 13-cis retinoic acid, hydrocortisone, hydrocortisone 21-acetate, hydrocortisone 17-valerate, hydrocortisone 17-butyrate, betamethasone valerate, betamethasone dipropionate, triamcinolone acetonide, fluocinonide, clobetasol propionate, benzoyl peroxide, crotamiton, propranolol, promethazine, vitamin A palmirate and vitamin E acetate.

Turning now to an additional feature of the present invention, the invention also provides an arrangement of projections which have antibacterial coatings to inhibit or reduce microbiological organism build up on and around the projections. This feature may be used to advantage with the dermabrasion and phototherapy devices of the present invention, but is also believed to be of patentable significance in a wide range of other applications of micro-projections or microneedles in the fields of abrasion, drug delivery, sampling and any other skin-interface application performed with micro-protrusions, particularly although not exclusively for applications in which the skin interface is either re-used or used for an extended period.

The term “anti bacterial” is used herein in the description and claims in a broad sense, to encompass any and all compounds, coatings or surface treatments effective to reduce the effects of microbiological contamination of a device, including, but not limited to, bacteria and other microorganisms (such as fungi). The antibacterial coating may kill bacteria, inhibit or reduce bacterial growth, and/or may reduce adherence of bacteria to surfaces of the device. Additional benefits of such materials and processes include the reduction or elimination of odors.

The anti-bacterial coating is preferably applied as a part of the manufacturing process of the skin-interface element. The coating is preferably chosen to be biocompatible, inexpensive and simple to manufacture. The anti-bacterial coating may be produced from materials such as: TITANIA (TiO2) and its derivatives, which are applied externally and later radiated with UV light (provided preferably in a separate lighting device). Titania coating is performed by well known techniques (e.g., sputtering, Chemical Vapor Deposition—CVD, MOCVD, deep coating etc.).

Additionally or alternatively, various metal ions could be used (or added to form combinations) such as Ag+, Zn2+, Co2+, Al3+ and Hg2+, and Cu3+. These materials have been shown to limit or inhibit bacterial growth. Again, they could be applied using techniques such as sputtering, deep coating, screen printing, painting, CVD, electroplating and other known techniques.

Other examples of coatings for imparting anti bacterial properties are a silver (and its derivatives) coating and BAK (Benzalkonium (BAK) chloride) coating. These materials do not require radiation for activation, and are know to exert anti bacterial properties. Additional materials include known chemical-based anti microbial agents (antibiotics).

The thickness of the anti-bacterial active layer is chosen according to the properties of the coating material used and the intended application, and lies in the range from a few nanometers up to hundreds of micrometers.

Optionally, the coating may also include a “replacement indicator” to indicate wearing out of a device or otherwise a need for a replacement. This indicator is preferably implemented as an internal paint layer which becomes revealed dependent on use or wearing (in a similar manner to shaving blade indicators). Such internal paint layers become noticeable shortly after mechanical wear reaches a predefined stage, thereby indicating that the skin interface element should be replaced.

It will be appreciated that the above descriptions are intended only to serve as examples, and that many other embodiments are possible within the scope of the present invention as defined in the appended claims.

What is claimed is:

1. A method for treatment of the skin comprising implementing a combined treatment including substantially contemporaneous or sequential steps of:
(a) performing dermabrasion to mechanically modify at least an outer layer of the skin in a first region; and
(b) delivering to said first region a therapeutically relevant dosage of light so as to perform phototherapy.

2. The method of claim 1, wherein said dermabrasion is performed prior to or during said delivering in such a manner as to reduce obstruction of the light passing through the outer layer of the skin.

3. The method of claim 1, wherein said dermabrasion is micro-dermabrasion.

4. The method of claim 1, wherein said dermabrasion is performed by bringing a plurality of micro-protrusions into contact with the skin and generating vibratory motion of the micro-protrusions.

5. The method of claim 4, wherein said micro-protrusions are provided with an antibacterial coating.

6. The method of claim 4, wherein at least part of a support structure supporting said micro-protrusions is formed from a material substantially transparent to said light, and wherein at least some of said light is delivered via said substantially transparent material.

7. The method of claim 1, further comprising, during said dermabrasion, delivering to said first region of skin a therapeutic substance.

8. The method of claim 7, wherein said therapeutic substance is an agent for enhancing action of said phototherapy.

9. A device for treatment of the skin comprising:
(a) a skin interface element including a substrate provided with a plurality of protrusions;
(b) a vibration generating mechanism mechanically linked to said skin interface element so as to generate vibratory motion of said skin interface element; and
(c) an illumination system deployed to direct a therapeutically relevant dosage of light towards a surface of the skin against which said skin interface unit is in contact.

10. The device of claim 9, further comprising a housing and a support structure supporting said skin interface element relative to said housing, wherein at least part of at least one of said substrate and said support structure is formed from a material substantially transparent to said light, said illumination system being deployed to direct at least some of said light via said substantially transparent material.

11. The device of claim 9, wherein said protrusions project to a height above said substrate of no greater than 200 microns;

12. The device of claim 9, wherein said protrusions project to a height above said substrate of between about 20 microns and about 100 microns.

13. The device of claim 9, wherein said protrusions are arranged in a two-dimensional array.

14. The device of claim 9, wherein said protrusions have a shape selected from the group comprising: pyramids, cones and rods.

15. The device of claim 9, wherein said protrusions are integrally formed with said substrate.

16. The device of claim 9, wherein said protrusions and said substrate are formed from a single crystal of material.

17. The device of claim 9, wherein said protrusions and said substrate are formed from a unitary block of material processed primarily by wet etching techniques.

18. The device of claim 9, wherein said protrusions are formed from a material selected from the group consisting of: silicon, a polymer, a metal, a metal alloy, and a ceramic material.

19. The device of claim 9, wherein said protrusions are provided with an antibacterial coating.

20. The device of claim 9, wherein said vibration generating mechanism includes a motor configured for rotating an eccentric weight about an axis.

21. The device of claim 9, wherein said vibration generating mechanism is configured to generate vibratory motion corresponding to an orbital motion in a plane of said substrate.

22. The device of claim 9, wherein said vibration generating mechanism is configured to generate vibratory motion having a non-zero component perpendicular to a plane of said substrate.

23. The device of claim 9, wherein said vibration generating mechanism is configured to generate vibratory motion having a frequency in the range between 50 Hz and 200 Hz.

24. The device of claim 9, wherein said vibration generating mechanism is configured to generate vibratory motion having a frequency in the range of 140 Hz±25 Hz.

25. The device of claim 9, further comprising a pressure-limiting switch arrangement associated with said skin interface element and responsive to contact pressure of said skin interface element above a given limit to interrupt operation of said vibration generating mechanism.

26. The device of claim 9, further comprising a housing, wherein said skin interface element is resiliently mounted relative to said housing, and wherein said vibration generating mechanism is mechanically linked to said skin interface element so as to generate vibratory motion of said skin interface element relative to said housing.

27. The device of claim 9, further comprising a housing mechanically supporting said skin interface element, said vibration generating mechanism, said illumination system and at least one electric battery, wherein said vibration generating mechanism and said illumination system are powered exclusively by said at least one electric battery.

28. A device for treatment of the skin comprising:
(a) a skin interface element including a substrate provided with a plurality of protrusions; and
(b) an antibacterial coating applied at least to surfaces of said protrusions.

29. The device of claim 28, wherein said antibacterial coating is applied to a surface of said substrate.

30. The device of claim 28, wherein said plurality of projections are implemented as a plurality of hollow microneedles.

31. The device of claim 28, wherein said antibacterial coating includes titanium dioxide.

32. The device of claim 28, wherein said antibacterial coating includes metal ions of at least one metal selected from the group consisting of: silver, zinc, cobalt, aluminum, mercury and copper.

33. The device of claim 28, wherein said antibacterial coating includes benzalkonium chloride.
